(19) United States
(12) Patent Application Publication
(10) Pub. No.: US 2026/0194692 A1
(43) Pub. Date: Jul. 9, 2026

(54) POLYSULFIDE RESIN, PRECURSOR, METHOD FOR PRODUCING POLYSULFIDE RESIN, OPTICAL ELEMENT, OPTICAL SYSTEM, LENS FOR INFRARED CAMERA, AND OPTICAL DEVICE

(71) Applicant: NIKON CORPORATION, Tokyo (JP)

(72) Inventors: Toshiki NAKANO, Kawasaki-shi (JP); Yohei KAYANO, Tokyo (JP); Akane KUBOTERA, Hiratsuka-shi (JP); Daisuke MORI, Hiratsuka-shi (JP); Yoshihiro SOMEYA, Yamato-shi (JP); Hideo TANAKA, Yokohama-shi (JP)

(73) Assignee: NIKON CORPORATION, Tokyo (JP)

(21) Appl. No.: 19/342,037

(22) Filed: Sep. 26, 2025

Related U.S. Application Data
(63) Continuation of application No. PCT/JP2024/001118, filed on Jan. 17, 2024.

(30) Foreign Application Priority Data
Mar. 29, 2023 (JP) 2023-053432

(51) Int. Cl. G02B 1/04; H04N 23/55

(52) U.S. Cl. CPC G02B 1/041; H04N 23/55

(57) ABSTRACT

A polysulfide resin in which structural units containing an adamantane structure are linked in a linear chain via sulfide bonds.

### TECHNICAL FIELD

The present invention relates to a polysulfide resin, a precursor, a method for producing a polysulfide resin, an optical element, an optical system, a lens for an infrared camera, and an optical device. This invention claims priority to Japanese Patent Application No. 2023-053432, filed on Mar. 29, 2023, and the contents of that application are incorporated by reference into this application in designated states where incorporation by reference is permitted.

### BACKGROUND ART

For example, an infrared camera equipped with a lens that focuses infrared light is known, as disclosed in PTL 1. Lens for such infrared cameras is required to be made from a low-cost resin material.
PTL 1: JP 2018-77072 A

### SUMMARY

A first aspect of the present invention is a polysulfide resin in which structural units including an adamantane structure are linked in a linear chain via sulfide bonds.

Another aspect of the present invention is a precursor of the above polysulfide resin, including a dithiol compound monomer and sulfur, and the ratio of the sulfur added in the precursor is 30 to 80 mass %.

Another aspect of the present invention is an optical element using the above polysulfide resin.

Another aspect of the present invention is an optical system including the above-described optical element.

Another aspect of the present invention is a lens for an infrared camera including the above-described optical system.

Another aspect of the present invention is an optical device including the above-described optical system.

Another aspect of the present invention is a method for producing a polysulfide resin, the method including: a mixing step of mixing a dithiol compound represented by a formula (2)

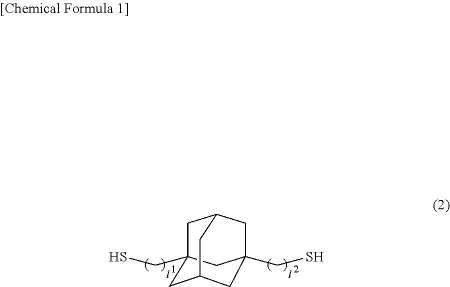

wherein l¹ and l² each independently represent an integer from 0 to 2 with sulfur in a molar ratio of 1:2.5 to 1:25 to obtain a mixture; and a reaction step of reacting the mixture at 120° C. to 220° C. for 12 to 48 hours.

### BRIEF DESCRIPTION OF THE DRAWINGS

FIG. 1 is a schematic view of an optical device according to the present embodiment used as a far-infrared digital camera;

FIG. 2 is a view illustrating the proportion of sulfide bonds in a polysulfide resin according to the present embodiment (monomer a: S addition ratios of 30, 50, and 70 mass %); and

FIG. 3 is a view illustrating the proportion of sulfide bonds in a polysulfide resin according to the present embodiment (monomers a, b, and c: S addition ratio of 50 mass %).

### DETAILED DESCRIPTION

The following describes in detail an embodiment of the present invention (hereinafter simply referred to as the “present embodiment”). The following present embodiment is an example for explaining the present invention and is not intended to limit the present invention to the following content.

### <Polysulfide Resin>

The polysulfide resin of the present embodiment is a novel resin in which structural units including an adamantane structure are linked in a linear chain via sulfide bonds. Such a resin can be suitably used as a component of materials such as optical elements for infrared cameras. Further, the use of such a resin enables the creation of optical elements with excellent far-infrared transmittance.

An example of a structural unit of the polysulfide resin according to the present embodiment is represented by the following formula (1).

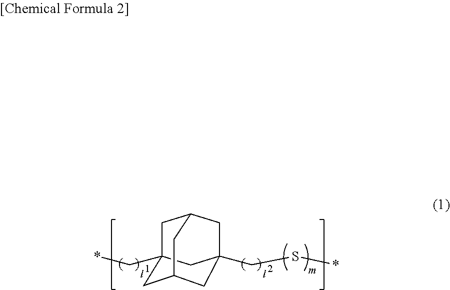

In the formula, l¹ and l² each independently represent an integer from 0 to 2.

The symbol * represents a bond and is responsible for the sulfide bond between the structural units represented by the formula (1).

The symbol m represents the number of sulfur atoms included in the formula (1) and is an integer of 2 or more. In this specification, a compound (1) in which m is 2 is referred to as a compound (i),

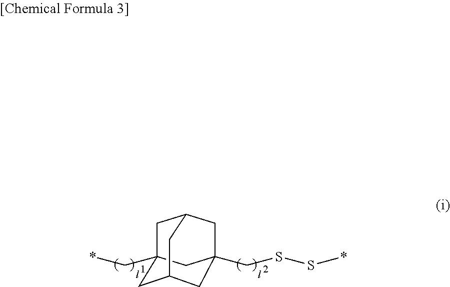

a compound (1) in which m is 3 is referred to as a compound (ii),

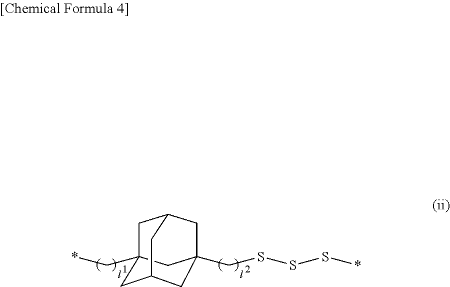

a compound (1) in which m is 4 or more is referred to as a compound (iii).

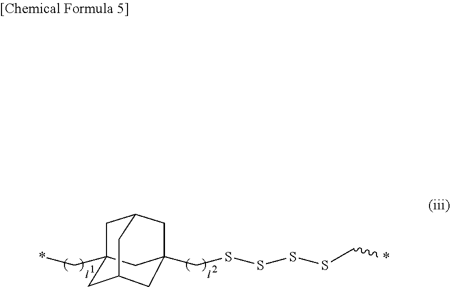

The wavy line in the formula for the compound (iii) outlines additional sulfide bonds when the compound (iii) includes more than four sulfur atoms.

The compounds (i), (ii), and (iii) are randomly linked in a linear chain via sulfide bonds via the bonds *.

The content ratio of the compound (i) where m is 2 in the polysulfide resin is 0 to 0.25 in terms of mole fraction. The lower limit of the content ratio of the compound (i) is preferably 0.02, more preferably 0.05, and still more preferably 0.08. The upper limit of the content ratio of the compound (i) is preferably 0.20, more preferably 0.10, and still more preferably 0.05.

The content ratio of the compound (ii) where m is 3 in the polysulfide resin is 0 to 0.25 in terms of mole fraction. The lower limit of the content ratio of the compound (ii) is preferably 0.04, more preferably 0.07, and still more preferably 0.10. The upper limit of the content ratio of the compound (ii) is preferably 0.20, more preferably 0.15, and still more preferably 0.10.

The content ratio of the compound (iii) where m is 4 or more in the polysulfide resin is 0.45 to 0.95 in terms of mole fraction. The lower limit of the compound (iii) content ratio is preferably 0.50, more preferably 0.55, and still more preferably 0.60. The upper limit of the content ratio of the compound (iii) is preferably 0.90, more preferably 0.85, and still more preferably 0.80.

### <Physical Properties of Polysulfide Resin>

The physical properties and the like of the polysulfide resin according to the present embodiment are described below.

The refractive index (n_{d}) of the polysulfide resin of the present embodiment with respect to the d-line is 1.60 to 1.95. The lower limit of the refractive index (n_{d}) may be 1.65, 1.67, or 1.70. The upper limit of the refractive index (n_{d}) may be 1.88, 1.86, or 1.84.

The external transmittance of the polysulfide resin of the present embodiment at wavelengths of 8 to 14 μm (far-infrared region) is 10% or more, preferably 15% or more, more preferably 20% or more, and still more preferably 24% or greater.

The polysulfide resin of the present embodiment has a film thickness of 0.3 to 2.0 mm. The lower limit of the film thickness is preferably 0.5 mm, more preferably 0.6 mm, and still more preferably 0.7 mm. The upper limit of the film thickness is preferably 2.0 mm, more preferably 1.8 mm, and still more preferably 1.5 mm.

### <Method for Producing Polysulfide Resin>

The polysulfide resin of the present embodiment is produced, for example, as shown below, by heating a precursor including a dithiol compound monomer of the formula (2) and solid sulfur to react with each other to produce a structural unit (1). The symbols in formulas (1) and (2) are as described above. The desired polysulfide resin may include trace amounts of elemental sulfur that precipitate during the production process.

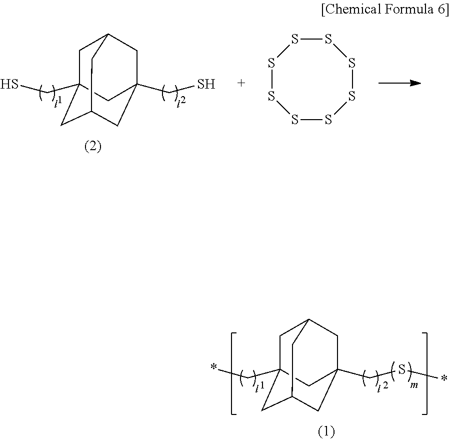

In one example of the method for producing a polysulfide resin according to the present embodiment, the sulfur addition ratio in a precursor including a dithiol compound monomer and sulfur is 30 to 85 mass %. The lower limit of the sulfur addition ratio is preferably 35%, more preferably 40%, and still more preferably 45%. The upper limit of the sulfur addition ratio is preferably 80%, more preferably 75%, and still more preferably 70%. If the sulfur addition ratio is less than 30%, a sufficient improvement in refractive index cannot be achieved. In contrast, when the sulfur addition ratio exceeds 80%, the cured resin lacks stability and sulfur precipitates over time.

One example of a method for producing a polysulfide resin according to the present embodiment includes a mixing step of mixing a dithiol compound monomer with sulfur in a molar ratio of 1:2.5 to 1:25 to obtain a mixture, and a reaction step of reacting the mixture at 120 to 220° C. for 12 to 48 hours. The molar ratio of the dithiol compound monomer to sulfur is represented as the number of sulfur atoms per monomer molecule.

The dithiol compound mixed in the mixing step preferably includes an adamantane structure, and more preferably is a dithiol compound such as those represented by the following formulas.

Monomer a (Wherein l¹ and l²=2)

[Chemical Formula 7]

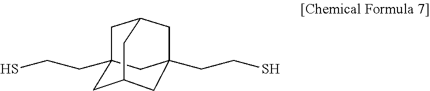 [SMILES: SCCC12CC3CC(C1)CC(CCS)(C3)C2]

Monomer b (Wherein l¹ and l²=1)

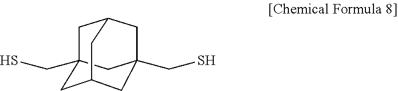 [SMILES: SCC12CC3CC(C1)CC(CS)(C3)C2]

Monomer c (Wherein l¹ and l²=0)

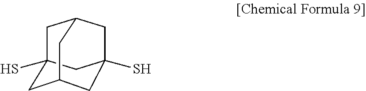 [SMILES: SC12CC3CC(C1)CC(S)(C3)C2]

The monomer a is synthesized, for example, by the following method.

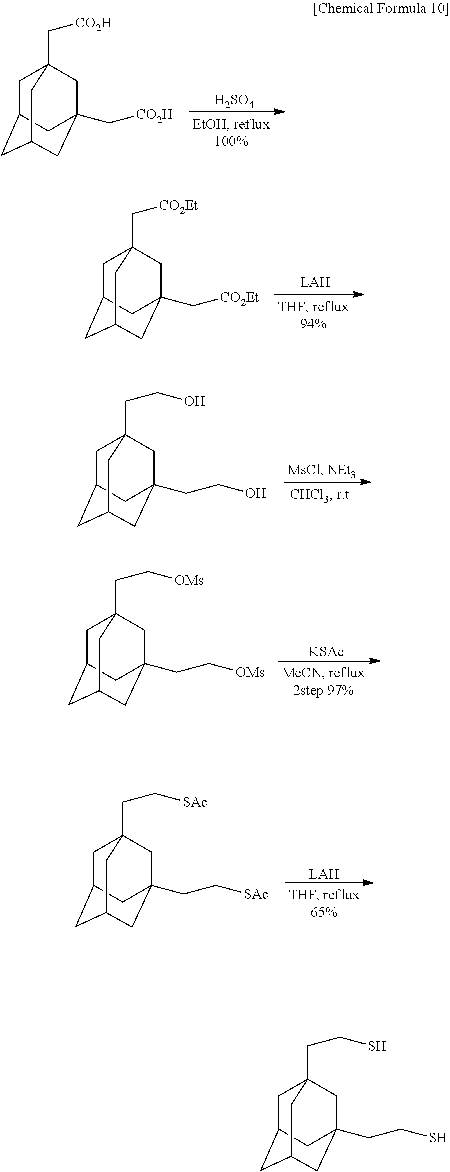

The monomer b is synthesized, for example, by the following method.

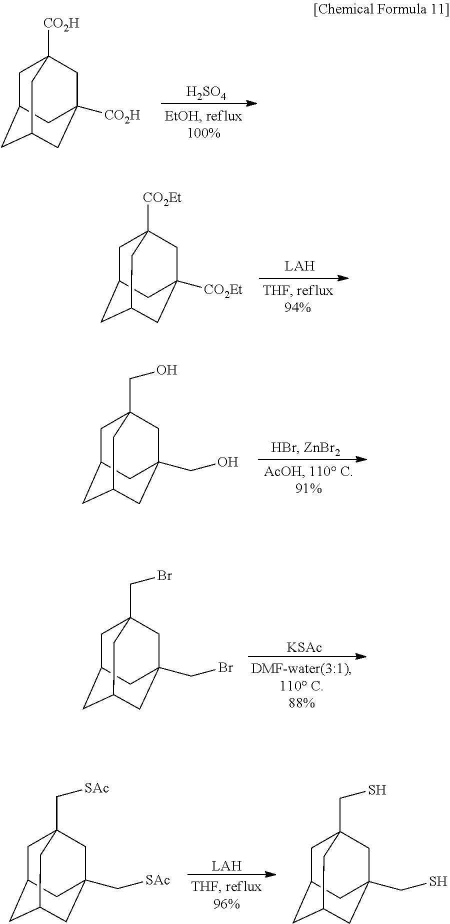

The monomer c is synthesized, for example, by the following method.

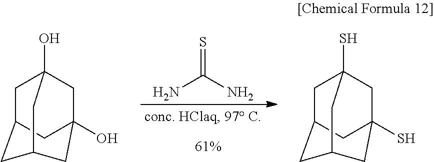

Details of the synthesis methods for the above monomers a, b, and c are described in the examples below. These monomers may also be synthesized by methods other than those described above, and the reaction steps during the synthesis may also be other than those described above, whether known or unknown.

The sulfur mixed in the mixing step is in the form of a powder element, and is commercially available from, for example, Sigma-Aldrich Co. LLC.

The molar ratio of the dithiol compound of the formula (2) to sulfur mixed in the mixing step is 1:2.5 to 1:25. When the dithiol compound is monomer a, the molar ratio is preferably 1:5.0, more preferably 1:8.0, and still more preferably 1:12.0. When the dithiol compound is monomer b, the molar ratio is preferably 1:3.5, more preferably 1:7.0, and still more preferably 1:10.5. When the dithiol compound is monomer c, the molar ratio is preferably 1:6.0, more preferably 1:10.0, and still more preferably 1:14.5.

The reaction temperature in the reaction step is 120 to 220° C. The lower limit of the reaction temperature is preferably 140° C., more preferably 150° C., and still more preferably 160° C. The upper limit of the reaction temperature is preferably 220° C., more preferably 210° C., and still more preferably 200° C.

The reaction time in the reaction step is 12 to 48 hours. The lower limit of the reaction time is preferably 16 hours, more preferably 20 hours, and still more preferably 24 hours. The upper limit of the reaction time is preferably 48 hours, more preferably 36 hours, and still more preferably 24 hours.

After the reaction step, the desired polysulfide resin can be obtained by allowing the resin to cool at room temperature for 1 to 4 hours. The cooling period is preferably 1 hour or more, more preferably 2 hours or more, and still more preferably 4 hours or more. The polysulfide resin according to the present embodiment may include a trace amount of elemental sulfur.

Other components, such as known clarifiers, colorants, defoamers, and fluorine compounds, can be added to the resin composition in appropriate amounts as needed for purposes such as clarification, coloring, decolorization, and fine adjustment of optical constants. In addition, other components, not limited to those listed above, can be added as long as the desired effects of the polysulfide resin according to the present embodiment can be obtained.

It is preferable to use a high-purity product with low impurity content as the raw materials. A high-purity product is one that includes 99.85 mass % or more of the relevant component. Using the high-purity product reduces the amount of impurities, which tends to increase the internal transmittance of the polysulfide resin.

### <Use of Polysulfide Resin>

From the viewpoint of the above, the polysulfide resin according to the present embodiment can be suitably used, for example, as an optical element in optical devices. Examples of such optical elements include mirrors, lenses, prisms, and filters. In addition, examples of the optical systems in which the above optical elements are used include objective lenses, condenser lenses, imaging lenses, and camera lenses. These optical systems can be suitably used in various optical devices such as far-infrared cameras (8 to 14 μm), mid-infrared cameras (3 to 5 μm), and near-infrared cameras (0.7 to 2.5 μm).

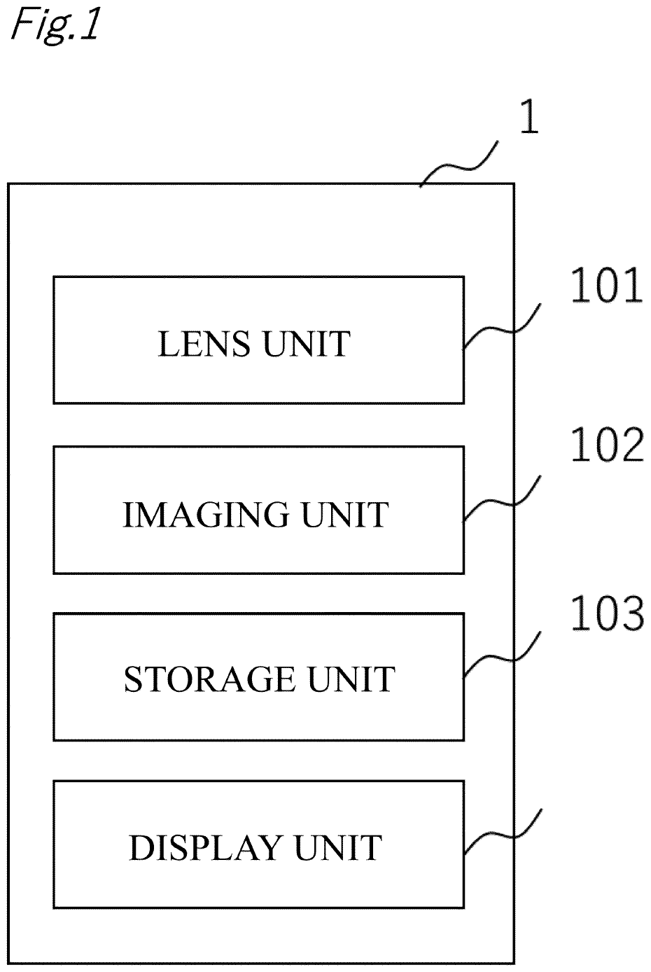

FIG. 1 is a schematic view of a far-infrared digital camera 1. The far-infrared digital camera 1 includes a lens unit 101, an imaging unit 102 equipped with a CMOS image sensor or the like, a storage unit 103 for storing image data acquired by the imaging unit, a display unit 104 for displaying the captured image, and the like. The lens unit 101 can use the polysulfide resin according to the present embodiment as its optical system.

### EXAMPLES

The present invention will be described in more detail with reference to the following examples, but the present invention is not limited to these examples.

Example 1 relates to a polysulfide resin (A) produced from the monomer a, Example 2 relates to a polysulfide resin (B) produced from the monomer b, and Example 3 relates to a polysulfide resin (C) produced from the monomer c.

Details of each step in the synthesis of the polysulfide resin, as well as the results of ¹H-NMR (AVANCE III HD manufactured by Bruker Corporation) and ¹³C-NMR (nuclear magnetic resonance spectrometer AVANCE III HD500) measurements of the compounds produced in each step, are shown below. In addition, the physical properties of the obtained polysulfide resin are shown in Tables 1 to 4.

### Example 1: Synthesis of Polysulfide Resin (A)

### Synthesis of Monomer a

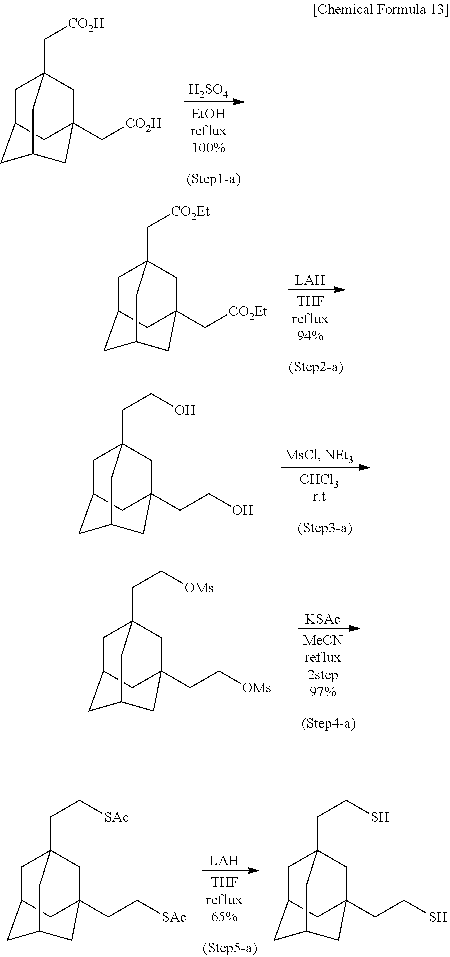

### Step 1-a

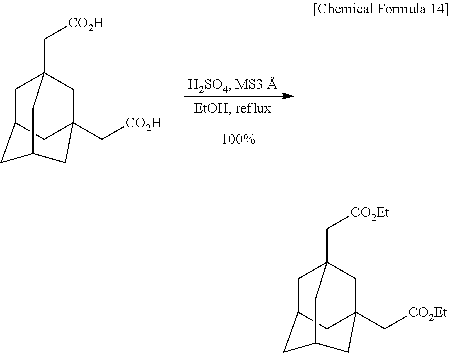 [SMILES: O=C(O)CC12CC3CC(C1)CC(CC(=O)O)(C3)C2 ; CCOC(=O)CC12CC3CC(C1)CC(CC(=O)OCC)(C3)C2]

An ethanol solution (100 mL) of 10.62 g (42.75 mmol) of 1,3-adamantanedicarboxylic acid (Tokyo Chemical Industry Co., Ltd.) was placed in a Soxhlet vessel packed with 3 Å molecular sieves, concentrated sulfuric acid (0.454 g, 4.629 mmol) was added, and the mixture was then heated to reflux for 20 hours. After cooling to room temperature, an aqueous NaHCO₃ solution (10 mL) was added and the mixture was concentrated under reduced pressure. The mixture was extracted with CHCl₃ (60 mL×3) and water (50 mL), and the organic layer was washed with saturated brine (30 mL). The organic layer was dried over Na₂SO₄, filtered, and the solvent was evaporated under reduced pressure to obtain the desired diester compound (13.19 g, 42.76 mmol) as a colorless, transparent liquid in 100% yield.

¹H-NMR (500 MHz, CDCl₃) δ4.11 (q, 4H, J=7.1 Hz), 2.12 to 2.17 (m, 2H), 2.02 (s, 2H), 1.81 to 1.90 (m, 8H), 1.66 to 1.69 (m, 2H), 1.24 (t, 6H, J=7.1 Hz); ¹³C-NMR (125 MHz, CDCl₃) δ177.7, 60.0, 48.6, 47.3, 41.6, 35.9, 33, 5, 29.0, 14.5

### Step 2-a

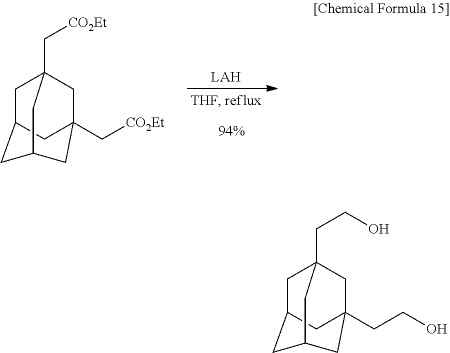 [SMILES: CCOC(=O)CC12CC3CC(C1)CC(CC(=O)OCC)(C3)C2 ; OCCC12CC3CC(C1)CC(CCO)(C3)C2]

Under a nitrogen atmosphere, anhydrous THF (20 mL) was added to LiAlH₄ powder (0.578 g, 7.619 mmol). The solution was cooled in an ice bath, and a THF solution (30 mL) of the diester compound (2.450 g, 7.619 mmol) was added. After heating to room temperature, the mixture was heated to reflux and stirred for 20 hours. After cooling to room temperature, the vessel was cooled in an ice bath and water (2 mL) was added dropwise. 20% aqueous NaOH solution (1 mL) was added, and the mixture was stirred at room temperature for 2 hours. The pH was adjusted to 3 to 4 with 6 M aqueous HCl solution, and the precipitated solid was filtered through a glass filter and washed with THF (50 mL). The solution was concentrated under reduced pressure, and the precipitated solid was dissolved in EtOAc (60 mL) by heating. The organic layer was extracted with saturated brine (20 mL), and the aqueous layer was extracted with EtOAc (20 mL). The combined organic layers were dried over Na₂SO₄, filtered, and the solvent was evaporated under reduced pressure to obtain the desired diol compound (6.80 g, 30.31 mmol) as a white solid in a yield of 94%.

¹H-NMR (500 MHz, CDCl₃) δ3.71 (t, 4H, J=7.5 Hz), 2.01 (t, 2H, J=2.91 Hz), 1.47 to 1.60 (m, 8H), 1.38 to 1.45 (m, 8H), 1.29 (s, 2H); ¹³C-NMR (125 MHz, CDCl₃) δ58.9, 48.3, 47.0, 42.3, 36.6, 32.7, 29.1

### Step 3-a

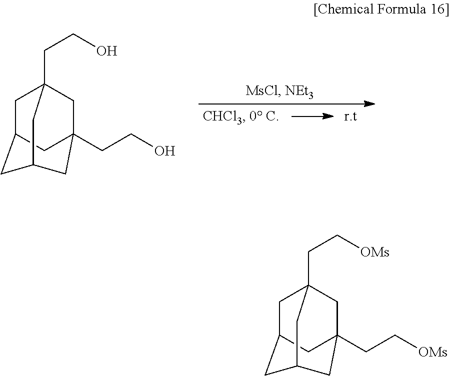 [SMILES: OCCC12CC3CC(C1)CC(CCO)(C3)C2 ; CS(=O)(=O)OCCC12CC3CC(C1)CC(CCOS(C)(=O)=O)(C3)C2]

A CHCl₃ solution (100 mL) of the diol compound (4.41 g, 19.65 mmol) was added, followed by triethylamine (13.6 mL, 98.1 mmol) and then mesyl chloride (6.1 mL, 78.8 mmol). After stirring at room temperature for 2 days, 1 M aqueous HCl solution (100 mL) was added, the mixture was extracted with CHCl₃ (50 mL×3), and the organic layer was washed with saturated brine (30 mL). The organic layer was dried over Na₂SO₄, filtered, and the solvent was evaporated under reduced pressure to obtain the crude dimesyl compound (9.91 g) as a pale yellow, transparent liquid. The crude product was used in the next step without purification (peak assignments are those at the time of purification). The product was liquid immediately after purification, but solidified after standing at room temperature for a while.

¹H-NMR (500 MHz, CDCl₃) δ4.29 (t, 4H, J=7.8 Hz), 3.00 (s, 6H), 2.06 (s, 2H), 1.49 to 1.62 (m, 10H), 1.41 to 1.47 (m, 4H), 1.31 (s, 2H); ¹³C-NMR (125 MHz, CDCl₃) δ66.4, 47.3, 42.4, 41.6, 37.5, 36.0, 32.5, 28.6

### Step 4-a

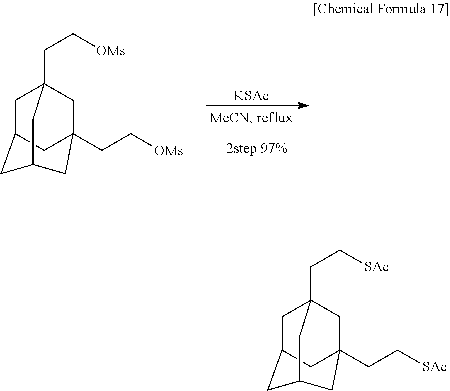

Potassium thioacetate (6.96 g, 60.94 mmol) was added to a MeCN solution (280 mL) of the crude dimesyl compound (9.91 g), and the mixture was heated to reflux and stirred for 24 hours. After cooling to room temperature, water (50 mL) was added and the solution was concentrated under reduced pressure. The mixture was extracted with EtOAc (60 mL×3) and water (30 mL), and the organic layer was washed with saturated brine (30 mL). The organic layer was dried over Na₂SO₄ and filtered. The solvent was evaporated under reduced pressure to obtain the crude product. The crude product was purified by column chromatography to obtain the desired dithioacetyl compound (6.517 g, 19.14 mmol) as an orange liquid in 97% yield (total yield for Step 3-a and Step 4-a).

¹H-NMR (500 MHz, CDCl₃) δ2.80 to 2.84 (m, 4H), 2.31 (s, 6H), 2.04 (s, 2H), 1.59 (s, 2H), 1.38 to 1.44 (m, 8H), 1.32 to 1.37 (m, 4H), 1.26 (s, 2H); ¹³C-NMR (125 MHz, CDCl₃) δ196.1, 46.7, 43.6, 41.6, 36.5, 33.7, 30.7, 29.0, 23.7

### Step 5-a

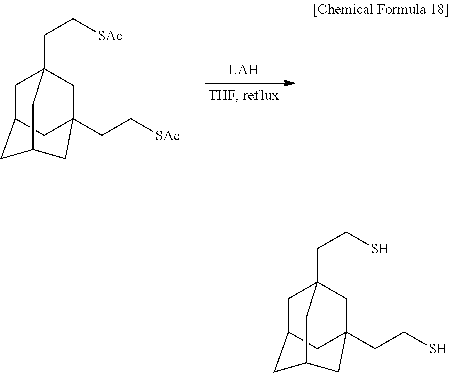

Under a nitrogen atmosphere, anhydrous THF (15 mL) was added to LiAlH₄ powder (1.45 g, 38.21 mmol). A THF solution (35 mL) of the dithioacetyl compound (6.52 g, 19.13 mmol) was added dropwise, and the mixture was heated to reflux for 20 hours. After cooling to room temperature, water (15 mL) and 2 M aqueous NaOH solution (5 mL) were added, and the mixture was stirred for 30 minutes. The solution was concentrated under reduced pressure, and the precipitated solid was filtered using a glass filter. The residue was washed with hot EtOAc (150 mL), and the organic layer was washed with saturated brine (20 mL) and 1 M aqueous HCl solution (20 mL). The aqueous layer was extracted with EtOAc (15 mL×2), and the combined organic layers were washed with saturated brine (20 mL). The organic layer was dried over Na₂SO₄ and filtered. The solvent was evaporated under reduced pressure to obtain the crude product. The crude product was purified by column chromatography to obtain the desired dithiol compound (3.20 g, 12.48 mmol) as a colorless, transparent liquid in a yield of 65% (monomer a).

¹H-NMR (500 MHz, CDCl₃) δ2.46 to 2.51 (m, 4H), 2.02 (s, 2H), 1.57 (s, 2H), 1.34 to 1.48 (m, 12H), 1.30 (t, 2H, J=2.4 Hz), 1.20 (s, 2H); ¹³C-NMR (125 MHz, CDCl₃) δ49.3, 47.0, 41.6, 36.4, 33.8, 28.8, 18.9

The monomer a and sulfur (Sigma-Aldrich Co. LLC) were weighed into a PFA reaction vessel such that the molar ratio and addition ratio were as shown in Table 1. The mixture was stirred at 200° C. for 3 to 6 hours. After stirring, the mixture was heated at 200° C. for an additional 18 to 21 hours (24 hours of heating in total) and then allowed to cool to room temperature to obtain a liquid. The liquid was then left to stand at room temperature for approximately 3 hours to obtain a polysulfide resin (1A to 5A).

### Example 2: Synthesis of Polysulfide Resin (B)

### Synthesis of Monomer b

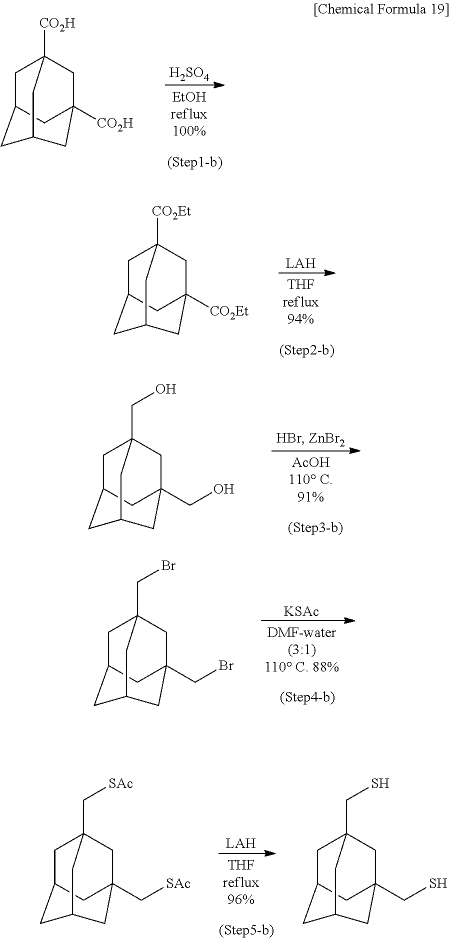

### Step 1-b

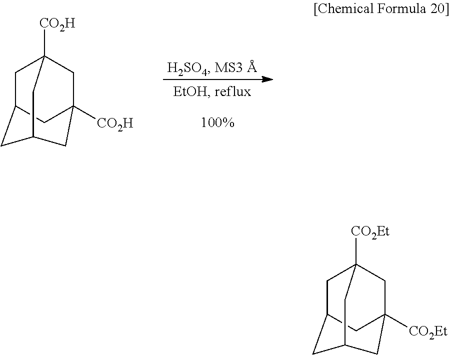 [SMILES: O=C(O)C12CC3CC(C1)CC(C(=O)O)(C3)C2 ; CCOC(=O)C12CC3CC(C1)CC(C(=O)OCC)(C3)C2]

An ethanol solution (220 mL) of 30.65 g (136.67 mmol) of 1,3-adamantanedicarboxylic acid (Tokyo Chemical Industry Co., Ltd.) was placed in a Soxhlet-equipped vessel packed with 3 Å molecular sieves, concentrated sulfuric acid (1.39 g, 14.17 mmol) was added, and the mixture was then heated to reflux for 22 hours. After cooling to room temperature, aqueous NaHCO₃ solution (30 mL) was added and the mixture was concentrated under reduced pressure. Extraction was performed with CHCl₃ (100 mL×3) and water (50 mL), and the organic layer was washed with saturated brine (30 mL). The organic layer was dried over Na₂SO₄ and filtered. The solvent was evaporated under reduced pressure to obtain the desired diester compound (38.2 g, 136.25 mmol) as a pale yellow, transparent liquid in 100% yield.

¹H-NMR (500 MHz, CDCl₃) δ3.25 (s, 4H), 2.11 (s, 2H), 1.65 (s, 2H), 1.42 to 1.54 (m, 10H), 1.29 (s, 2H); ¹³C-NMR (125 MHz, CDCl₃) δ73.6, 40.6, 38.8, 36.8, 35.2, 28.3

### Step 2-b

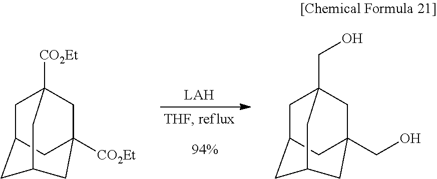 [SMILES: CCOC(=O)C12CC3CC(C1)CC(C(=O)OCC)(C3)C2 ; OCC12CC3CC(C1)CC(CO)(C3)C2]

Under a nitrogen atmosphere, anhydrous THF (30 mL) was added to LiAlH₄ powder (3.81 g, 100.4 mmol). The solution was cooled in an ice bath, and a THF solution (150 mL) of the diester compound (14.02 g, 50.01 mmol) was added using a dropping funnel. After dropping was complete, the temperature was raised to room temperature and the mixture was heated to reflux and stirred for 24 hours. After allowing the mixture to cool to room temperature, the vessel was cooled in an ice bath and water (10 mL) was added dropwise. 10% aqueous NaOH solution (5 mL) was added and the mixture was stirred at room temperature for 30 minutes. The pH was adjusted to 4 to 5 with 2 M aqueous HCl solution, and the precipitated solid was filtered through a glass filter. The solution was then concentrated under reduced pressure. The residue was washed with THF (100 mL) and concentrated under reduced pressure. The mixture was extracted with hot EtOAc (100 mL×3) and water (100 mL), and the mother liquor was washed with saturated brine (20 mL). The organic layer was dried over Na₂SO₄ and filtered. The solvent was evaporated under reduced pressure to obtain the desired 1,3-adamantanedimethanol (9.27 g, 47.2 mmol) as a white solid in a 94% yield.

¹H-NMR (500 MHz, CDCl₃) δ3.25 (s, 4H), 2.11 (s, 2H), 1.65 (s, 2H), 1.42 to 1.54 (m, 10H), 1.29 (s, 2H); ¹³C-NMR (125 MHz, CDCl₃) δ73.6, 40.6, 38.8, 36.8, 35.2, 28.3

### Step 3-b

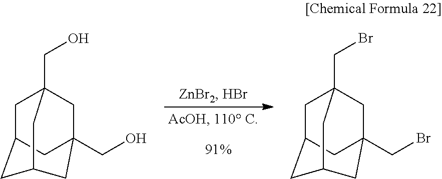 [SMILES: OCC12CC3CC(C1)CC(CO)(C3)C2 ; BrCC12CC3CC(C1)CC(CBr)(C3)C2]

ZnBr₂ (5.63 g, 25.00 mmol) and a 30% HBr—AcOH solution (15 mL) were added to 1,3-adamantanedimethanol (0.981 g, 5.00 mmol), and the mixture was stirred at room temperature for 15 minutes, followed by stirring at 110° C. for 5 hours. After allowing the mixture to cool to room temperature, the mixture was left to stand overnight. The precipitated solid was filtered using a glass filter, and the residue was washed with water (30 mL×3). After drying under reduced pressure, the desired 1,3-dimethylenebromoadamantane (1.46 g, 4.53 mmol) was obtained as a white solid in a 91% yield.

¹H-NMR (500 MHz, CDCl₃) δ 3.19 (2, 4H), 2.12 (s (br), 2H), 1.58 (s (br), 2H), 1.46 to 1.55 (m, 10H); 13C-NMR (125 MHz, CDCl₃) δ47.4, 43.8, 40.1, 35.9, 34.6, 28.7

### Step 4-b

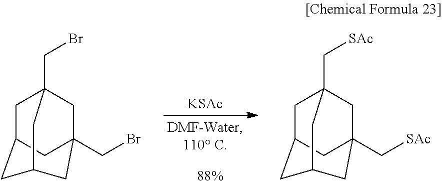

Potassium thioacetate (1.55 g, 13.57 mmol) was added to a solution of 1,3-dimethylenebromoadamantane (1.457 g, 4.524 mmol) in DMF-water (4:1, 30 mL) and stirred at 110° C. for 18 hours. After cooling to room temperature, water (70 mL) was added and the mixture was extracted with Et₂O (30 mL×3). The organic layer was washed with saturated brine (15 mL×2). The organic layer was dried over Na₂SO₄ and filtered. The solvent was evaporated under reduced pressure to obtain a crude product. The product was isolated and purified by column chromatography to obtain the desired dithioacetyl compound (1.247 g, 3.991 mmol) as an orange liquid in 88% yield.

¹H-NMR (500 MHz, CDCl₃) δ2.72 (s, 4H), 2.32 (s, 6H), 2.01 (s, 2H), 1.53 (s, 2H), 1.33 to 1.46 (m, 8H), 1.22 (s, 2H); ¹³C-NMR (125 MHz, CDCl₃) δ195.7, 45.2, 41.9, 40.6, 35.9, 34.1, 30.8, 28.7

### Step 5-b

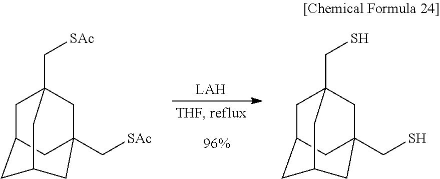

Under a nitrogen atmosphere, anhydrous THF (10 mL) was added to LiAlH₄ powder (0.227 g, 5.982 mmol), a solution of the dithioacetyl compound (0.932 g, 2.982 mmol) in THF (20 mL) was added dropwise, and heated to reflux for 20 hours. After cooling to room temperature, water (2 mL), 15% aqueous NaOH solution (1 mL), and THF (10 mL) were added and stirred at room temperature for 1.5 hours. After adjusting the pH to 3 to 4 with 6 M aqueous HCl solution, the precipitate was filtered through a glass filter and the residue was washed with THF (50 mL). The solution was concentrated under reduced pressure and then diluted with EtOAc (80 mL), and the organic layer was washed with saturated brine (15 mL). The organic layer was dried over Na₂SO₄ and filtered. The solvent was evaporated under reduced pressure to obtain a crude product. Isolation and purification by column chromatography provided the desired dithiol compound (0.657 g, 2.876 mmol) as a colorless, transparent liquid in a 96% yield (monomer b).

¹H-NNR (500 MHz, CDCl₃) δ2.34 (d, 4H, J=8.7 Hz), 2.10 (m, 2H), 1.39 to 1.51 (m, 8H), 1.28 (s, 2H), 1.33 to 1.46 (m, 8H), 1.11 (t, 2H, J=8.7 Hz); ¹³C-NMR (125 MHz, CDCl₃) δ44.4, 40.5, 38.3, 36.3, 34.2

The monomer b and sulfur (Sigma-Aldrich Co. LLC) were weighed into a PFA reaction vessel such that the molar ratio and addition ratio were as shown in Table 2. This mixture was stirred at 200° C. for 3 to 6 hours. After stirring, the mixture was heated at 200° C. for an additional 18 to 21 hours (a total of 24 hours of heating), then allowed to cool to room temperature to obtain a liquid. The liquid was then allowed to stand at room temperature for approximately 3 hours to obtain a polysulfide resin (1B to 4B).

### Example 3: Synthesis of Polysulfide Resin (C)

### Synthesis of Monomer c

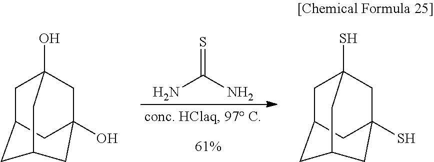

Thiourea (906 g, 11.9 mol) was added to a 10 L four-neck flask including 1,3-adamantanediol (100 g, 0.6 mol) (Tokyo Chemical Industry Co., Ltd.) in concentrated aqueous hydrochloric acid (approximately 12 M, 3 kg, 30 W equivalent), and the mixture was heated and stirred overnight at an internal temperature of 97° C. (the reaction solution changed from a white suspension to a colorless solution, and a white solid precipitated on the walls of the reactor. As much of the precipitated solid as possible was scraped off with a spatula and dropped into the reaction solution). After allowing the mixture to cool to room temperature, 48% aqueous NaOH solution (1.6 L) was added dropwise over 1 hour (internal temperature: maximum 39° C.) to adjust the pH to 12. The reaction solution was then heated to an internal temperature of 97° C. and stirred for 1 hour. After allowing the reaction solution to cool to room temperature, the pH was adjusted to 1 by adding concentrated aqueous hydrochloric acid (200 mL) dropwise while cooling in an ice bath. CHCl₃ (3 L) was added and the mixture was stirred, and the precipitated solid (thiourea, its residue, and NaCl) was filtered. The filtered solid was washed with CHCl₃ (1 L×3), and the organic layer combined with the mother liquor was washed with water (5 L) and then saturated brine (5 L). The organic layer was concentrated to obtain a crude solid (1).

This crude solid (1) was transferred back to the reactor while being washed with CHCl₃ (230 mL). To this were added thiourea (906 g, 11.9 mol) and concentrated aqueous hydrochloric acid (approximately 12 M, 3 kg, 30 w equivalent), and the mixture was heated and stirred at an internal temperature of 97° C. CHCl₃, which had been started to be refluxed, was removed as much as possible using a Dean-Stark flask (distillate volume: 140 mL), and after heating and stirring overnight, the reaction solution was allowed to cool. While cooling the reaction solution in an ice bath, 48% aqueous NaOH solution (1.7 L) was added dropwise over 90 minutes (internal temperature: maximum 39° C.) to adjust the pH to 12. The reaction solution was then heated to an internal temperature of 97° C. and stirred for 3 hours. After allowing the reaction solution to cool to room temperature, concentrated aqueous hydrochloric acid (330 mL) was added dropwise while cooling in an ice bath to adjust the pH to 2. CHCl₃ (3 L) was added, the mixture was stirred, and the precipitated solid (thiourea, its residue, and NaCl) was filtered. The filtered solid was washed with CHCl₃ (1 L×3), and the organic layer combined with the mother liquor was washed with water (5 L) and then saturated brine (5 L). The organic layer was concentrated to obtain a crude solid (2).

The crude solid (2) was isolated and purified by column chromatography to obtain the desired 1,3-adamantanedithiol compound (73.1 g, 0.365 mol) as a white solid in a 61% yield (monomer c).

¹H-NMR (500 MHz, CDCl₃) δ 2.14 (s, 2H), 2.08 (s, 2H), 1.83 (m, 8H), 1.73 (s, 2H), 1.58 (s, 2H); ¹³C-NMR (125 MHz, CDCl₃) δ 57.3, 45.8, 43.9, 34.2, 31.6

The monomer c may be synthesized by the following method.

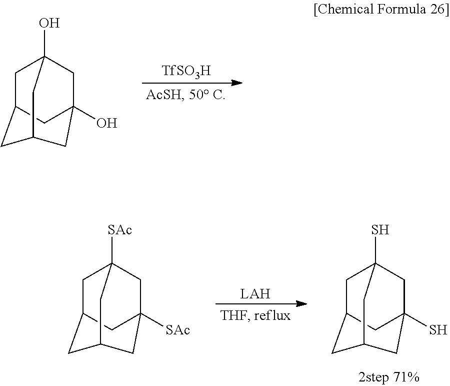

Trifluoromethanesulfonic acid (0.52 mL, 5.89 mmol) was added to a solution of 1,3-adamantanediol (9.943 g, 59.10 mmol) in thioacetic acid (25 mL), and the mixture was stirred at 50° C. for 22 hours. After cooling to room temperature, the mixture was diluted with CHCl₃ (150 mL) and washed with aqueous NaHCO₃ solution (100 mL×3), and the aqueous layer was washed with CHCl₃ (70 mL×2). The combined organic layer was washed with saturated brine (30 mL×2). The organic layer was dried over Na₂SO₄ and filtered. The solvent was evaporated under reduced pressure to obtain the crude product (19.8 g). The crude product was used in the subsequent step without purification.

Under a nitrogen atmosphere, anhydrous THF (100 mL) was added to LiAlH₄ powder (6.77 g, 178.4 mmol). While the vessel was cooled in an ice bath, an anhydrous THF solution (80 mL) of the crude product described above was added dropwise. After heating to room temperature, the mixture was heated to reflux and stirred for 20 hours. After allowing the mixture to cool to room temperature, the vessel was cooled in an ice bath, and water (12 mL) and 20% aqueous NaOH solution (6 mL) were added dropwise, in this order. THF (150 mL) was then added, and the mixture was stirred at room temperature for 1 hour. After adjusting the pH to 3 to 4 with 12 M aqueous HCl solution, the precipitated solid was filtered through a glass filter, and the residue was washed with THF (250 mL). The solution was concentrated under reduced pressure, diluted with EtOAc (300 mL), and washed with water (100 mL) and saturated brine (50 mL), in this order. The organic layer was dried over Na₂SO₄ and filtered. The solvent was evaporated under reduced pressure to obtain a crude product. This was purified by column chromatography (hexane/toluene) to obtain the desired 1,3-adamantanedithiol (8.461 g, 42.23 mmol) in a two-step yield of 71% (monomer c).

The monomer c and sulfur (Sigma-Aldrich Co. LLC) were weighed into a PFA reaction vessel such that the molar ratio and addition ratio were as shown in Table 3. This mixture was stirred at 200° C. for 3 to 6 hours. After stirring, the mixture was heated at 200° C. for an additional 18 to 21 hours (a total of 24 hours of heating), and then allowed to cool to room temperature to obtain a liquid. This liquid was then allowed to stand at room temperature for approximately 3 hours to obtain a polysulfide resin (1C to 3C).

### <Evaluation of Polysulfide Resin Properties>

The refractive index (n_{d}), far-infrared transmittance, and film thickness were measured for the resulting polysulfide resins A (1A to 5A), B (1B to 4B), and C (1C to 3C). In addition, the refractive index and transmittance in the terahertz range were measured for polysulfide resins 3A, 2B, 1C, and 3C.

The refractive index n_{d} was measured for the d-line (587.6 nm) using a prism coupler (Metricon Corporation). The refractive index in the terahertz range was measured using time-domain spectroscopy (THz-TDS) with a terahertz spectroscopic imaging device (prototype, measurement frequency band: 0.1 to 1.5 THz, frequency resolution: approximately 5 GHz, spatial resolution: approximately 100 μm). In addition to the transmittance spectrum described below, the phase difference spectrum was measured simultaneously, and the refractive index spectrum was derived from these results (Reference: K. Sakai, “Terahertz Optoelectronics,” Topics Appl. Phys. 97, 203 (2005)).

Far-infrared transmittance was measured using the transmission method from 4000 to 400 cm⁻¹ using an FT-IR (Nicolet 6700 manufactured by Thermo Fisher Scientific Inc.). The resolution was 2, and the number of accumulations was 30. The average transmittance was calculated for a wavelength range of 8 to 14 μm. The transmittance was calculated by averaging the integrated values obtained using the trapezoidal rule, with the unit kept as wavenumber (cm⁻¹). The refractive index in the terahertz range was measured using time-domain spectroscopy (THz-TDS) with a terahertz spectroscopic imaging device (prototype). Spectroscopic spectra were acquired using spectroscopic imaging, and transmittance was derived by comparing them with reference data (Teflon (registered trademark) substrate).

The film thickness was measured at the center using a constant pressure thickness gauge (PG-01 manufactured by TECLOCK Corporation) (the thickness was also measured at the periphery (four locations) to confirm uniformity). The above-described measuring device complies with JIS K6250.

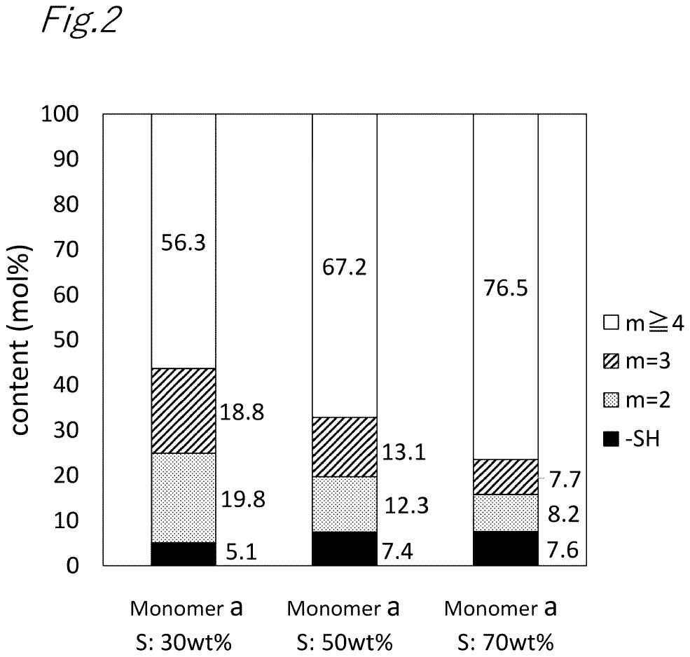

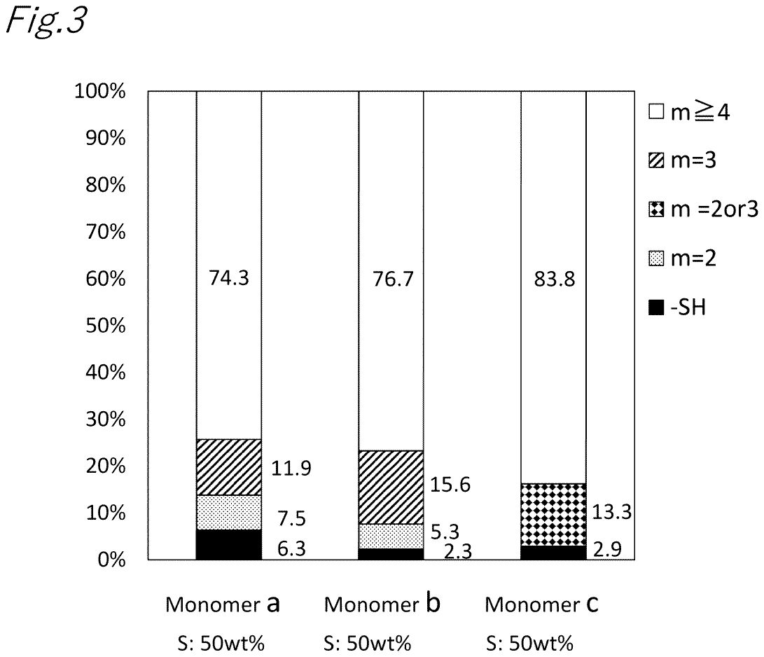

The measurement results for the polysulfide resin A are shown in Example 1 of Table 1 (Examples 1-1 to 1-5), the measurement results for the polysulfide resin B in Example 2 of Table 2 (Examples 2-1 to 2-4), and the measurement results for the polysulfide resin C in Example 3 of Table 3 (Examples 3-1 to 3-3). Table 4 shows the measurement results for refractive index and transmittance in the terahertz range. FIGS. 2 and 3 show the proportion of sulfide bonds (abundance ratio of m numbers) in polysulfide resins when ratios of the monomer and sulfur added are varied. The abundance ratio of m numbers was calculated from the results of ¹H-NMR (500 MHz, CDCl₃). Taking the monomer a as an example, the peak derived from —CH₂SH is detected at δ=2.48, δ=2.58 to 2.65 for m=2, δ=2.85 for m=3, and δ=2.97 for m≥4. The m-number abundance ratio was calculated by using the sum of the integral ratios of these peaks as the denominator and the integral ratio of the corresponding peak as the numerator. For the monomer b, the exact peak positions were different, but the calculation was similar. For the monomer c, in addition to an overall peak position shift, the peak positions of m=2 and m=3 overlapped, and thus separation was not performed.

TABLE 1

| | | | | | Far- | |
|---|---|---|---|---|---|---|
| | | | Reaction | | infrared | |
| | | S | temper- | Refrac- | average | Film |
| | Molar | addition | ature | tive | trans- | thick- |
| | ratio | ratio | and time | index | mittance | ness |
| | (m:s) | (wt %) | (° C., h) | (n_{d}) | (%) | (mm) |
| Example 1-1 | 1:3.4 | 30 | (180, 24) | 1.67 | 1.26 | 1.2 |
| (1A) | | | | | | |
| Example 1-2 | 1:3.4 | 30 | (200, 24) | 1.67 | 0.94 | 1.2 |
| (2A) | | | | | | |
| Example 1-3 | 1:8.0 | 50 | (200, 24) | 1.72 | 3.08 | 0.7 |
| (3A) | | | | | | |
| Example 1-4 | 1:12.0 | 60 | (200, 24) | 1.74 | 2.24 | 0.8 |
| (4A) | | | | | | |
| Example 1-5 | 1:18.7 | 70 | (200, 24) | 1.77 | 0.41 | 1.5 |
| (5A) | | | | | | |

TABLE 2

| | | | | | Far- | |
|---|---|---|---|---|---|---|
| | | | Reaction | | infrared | |
| | | S | temper- | Refrac- | average | Film |
| | Molar | addition | ature | tive | trans- | thick- |
| | ratio | ratio | and time | index | mittance | ness |
| | (m:s) | (wt %) | (° C., h) | (n_{d}) | (%) | (mm) |
| Example 2-1 | 1:3.1 | 30 | (200, 24) | 1.69 | 3.8 | 1.1 |
| (1B) | | | | | | |
| Example 2-2 | 1:7.1 | 50 | (200, 24) | 1.76 | 7.16 | 0.9 |
| (2B) | | | | | | |
| Example 2-3 | 1:10.7 | 60 | (200, 24) | 1.76 | 8.21 | 0.8 |
| (3B) | | | | | | |
| Example 2-4 | 1:16.6 | 70 | (200, 24) | 1.79 | 12 | 0.7 |
| (4B) | | | | | | |

TABLE 3

| | | | | | Far- | |
|---|---|---|---|---|---|---|
| | | | Reaction | | infrared | |
| | | S | temper- | Refrac- | average | Film |
| | Molar | addition | ature | tive | trans- | thick- |
| | ratio | ratio | and time | index | mittance | ness |
| | (m:s) | (wt %) | (° C., h) | (n_{d}) | (%) | (mm) |
| Example 3-1 | 1:6.3 | 50 | (200, 24) | 1.79 | 11.1 | 0.9 |
| (1C) | | | | | | |
| Example 3-2 | 1:14.6 | 70 | (200, 24) | 1.81 | 17.4 | 1 |
| (2C) | | | | | | |
| Example 3-3 | 1:25.0 | 80 | (200, 24) | 1.82 | 23.6 | 0.6 |
| (3C) | | | | 1.84 | 22.4 | 0.7 |

TABLE 4

| | | | Reaction | Refrac- | 0.1 THz | |
|---|---|---|---|---|---|---|
| | | S | temper- | tive | average | Film |
| | Molar | addition | ature | index | trans- | thick- |
| | ratio | ratio | and time | (0.1 | mittance | ness |
| | (m:s) | (wt %) | (° C., h) | THz) | (%) | (mm) |
| Example 1-3 | 1:8.0 | 50 | (200, 24) | 1.71 | 82.7 | 1.5 |
| (3A) | | | | | | |
| Example 2-1 | 1:3.1 | 30 | (200, 24) | 1.69 | 89.7 | 1.5 |
| (1B) | | | | | | |
| Example 2-2 | 1:7.1 | 50 | (200, 24) | 1.70 | 85.2 | 1.5 |
| (2B) | | | | | | |
| Example 3-1 | 1:6.3 | 50 | (200, 24) | 1.70 | 88.9 | 1.5 |
| (1C) | | | | | | |
| Example 3-3 | 1:25.0 | 80 | (200, 24) | 1.88 | 90.8 | 1.5 |
| (3C) | | | | | | |

As shown in Tables 1 to 3, it was confirmed that the refractive index and far-infrared transmittance of the polysulfide resins of Examples 1 to 3 increased with an increase in the sulfur addition ratio.

### REFERENCE SIGNS LIST

1 Far-infrared digital camera
101 Lens unit
102 Imaging unit
103 Storage unit
104 Display unit

1. A polysulfide resin wherein structural units including an adamantane structure are linked in a linear chain via sulfide bonds.

2. The polysulfide resin according to claim 1, wherein the structural unit is represented by the formula (1): 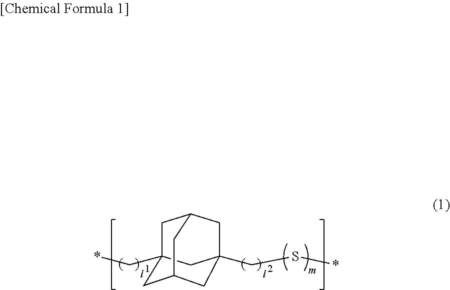
wherein l¹ and l² each independently represent an integer of 0 to 2, m represents an integer of 2 or more, and * represents a bond.

3. The polysulfide resin according to claim 2, wherein content ratios of a compound i where m is 2, a compound ii where m is 3, and a compound iii where m is 4 or more are, in mole fractions, as follows:
compound i: 0 to 0.25,
compound ii: 0 to 0.25, and
compound iii: 0.45 to 0.95.

4. The polysulfide resin according to claim 1, wherein a refractive index (n_{d}) is 1.60 to 1.95.

5. The polysulfide resin according to claim 1, wherein a transmittance in a far-infrared region (wavelengths of 8 to 14 μm) is 10% or more.

6. The polysulfide resin according to claim 1, wherein a thickness is 0.3 to 2.0 mm.

7. A precursor of the polysulfide resin according to claim 1, comprising:
a monomer of a dithiol compound; and
sulfur,
wherein a ratio of the sulfur added in the precursor is 30 to 80 mass %.

8. An optical element using the polysulfide resin according to claim 1.

9. An optical system including the optical element according to claim 8.

10. A lens for an infrared camera, comprising the optical system according to claim 9.

11. An optical device comprising the optical system according to claim 9.

12. A method for producing a polysulfide resin, the method comprising:
a mixing step of mixing a dithiol compound represented by the formula (2): 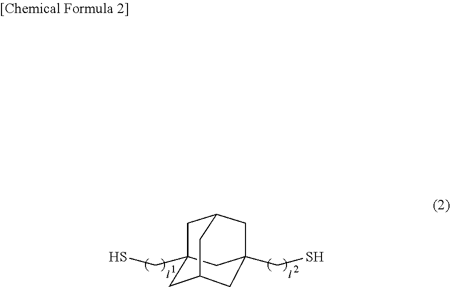
wherein l¹ and l² each independently represent an integer of 0 to 2 with sulfur in a molar ratio of 1:2.5 to 1:25 to obtain a mixture; and
a reaction step of reacting the mixture at 120 to 220° C. for 12 to 48 hours.
